(19) United States
(12) Patent Application Publication
(10) Pub. No.: US 2011/0275145 A1
(43) Pub. Date: Nov. 10, 2011

(54) MANUFACTURING METHOD AND STRUCTURE OF CELL CRYOPRESERVATION TUBE

(76) Inventors: Wei-Te WU, Taichung City (TW); Hsun-Heng Tsai, Tainan City (TW)

(21) Appl. No.: 12/773,632

(22) Filed: May 4, 2010

(51) Int. Cl. C12M 1/24; B21D 3/00

(52) U.S. Cl. 435/307.1; 72/367.1

(57) ABSTRACT

Manufacturing method of cell cryopreservation tube comprises the steps of providing a metal tubule having an inside wall and an outside wall. Smoothing the inside wall of the metal tubule thereby forming a smooth surface on the inside wall, and the roughness of the smooth surface is named the first roughness. Roughening the outside wall of the metal tubule thereby forming a rough surface on the outside wall, and the roughness of the rough surface is called the second roughness. The magnitude of the second roughness is greater than the first roughness.

### FIELD OF THE INVENTION

The present invention is generally relating to a manufacturing method and structure of cell cryopreservation tube.

### BACKGROUND OF THE INVENTION

A conventional cell cryopreservation tube typically utilizes a quartz tube, glass tube or polymer tube. Therefore, the quartz tube and glass tube likely to crack under low temperature operating environment. The polymer tube is capable of operating under low temperature, but the defect of relative low-thermal conduction thereby enabling the appearance of intracellular ice damage from the cell.

### SUMMARY

A primary object of the present invention is to offer a manufacturing method and structure of cell cryopreservation tube comprising the steps of providing a metal tubule having an inside wall and an outside wall. Smoothing the inside wall of the metal tubule thereby forming a smooth surface on the inside wall, and the roughness of the smooth surface is named the first roughness. Roughening the outside wall of the metal tubule thereby forming a rough surface on the outside wall, and the roughness of the rough surface is called the second roughness. The magnitude of the second roughness is greater than the first roughness. This invention is capable of accelerating the low-thermal conduction conducted from outside to the inside of the metal tubule via the rough surface to prevent the formation of intracellular ice. The metal tubule possesses good heat conduction and is capable of operating under low temperature.

The secondary object of the present is to offer a structure of the cell cryopreservation tube. The cell cryopreservation tube is a metal tubule and has an inner wall having a smooth surface and an outer wall having a rough surface. The smooth surface has a first roughness, and the rough surface has a second roughness. The magnitude of the second roughness is greater than the first roughness.

### DESCRIPTION OF THE DRAWINGS

FIG. 1 is a manufacturing flow chart illustrating a cell cryopreservation tube in accordance with an embodiment of the present invention.

FIG. 2A-2C is an indicated view illustrating the cell cryopreservation tube in accordance with an embodiment of the present invention.

FIG. 3A-3C is a perspective view illustrating the cell cryopreservation tube in accordance with an embodiment of the present invention.

### DETAILED DESCRIPTION OF THE INVENTION

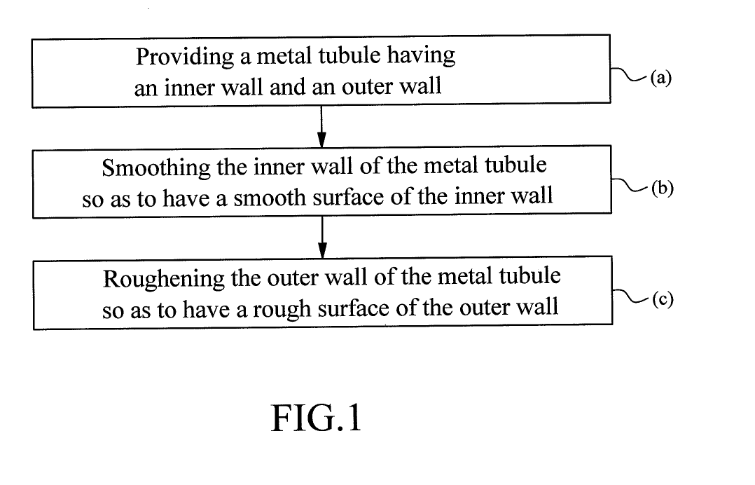

Referring to FIG. 1, 2A-2C and 3A-3C, a manufacturing method and structure of cell cryopreservation tube comprising the steps described as followed. First, referring to step (a) of FIG. 1, 2A and 3A, providing a metal tubule 10 having an inner wall 11 and an outer wall 12. In this embodiment, the material of the metal tubule 10 is one of the groups being composed of stainless steel and titanium; After that, referring to step (b) of FIG. 1, 2B and 3B, smoothing the inner wall 11 of the metal tubule 10 so as to have a smooth surface 11a of the inner wall 11 and the smooth surface 11a having a first roughness. In this embodiment, the method of smoothing the inner wall of the metal tubule is the method of electrolytic polishing. At last, referring to step (c) of FIG. 1, 2C and 3C, roughening the outer wall 12 of the metal tubule 10 so as to have a rough surface 12a of the outer wall 12 and the rough surface 12a having a second roughness. In this embodiment, the magnitude of the second roughness is greater than the first roughness. Further in this embodiment, the method of roughening the outer wall 12 of the metal tubule 10 is the method of electrochemical anodization treatment. After the step of smoothing the inner wall 11 of the metal tubule 10, it further includes another step of sealing two ends of the meal tubule 10 to prevent electrolyte from flowing into the inside of the metal tubule 10 to cause corrosion of the smooth surface 11a. Or, in another embodiment, the method of roughening the outer wall 12 of the metal tubule 10 is the method of grinding so as to ignore the step of sealing two ends of the metal tubule 10. This invention is capable of accelerating the low-thermal conduction conducted from outside to the inside of the metal tubule 10 via the rough surface 12a to prevent the formation of intracellular ice. The smooth surface 11a prevents low temperature radiant energy from conducting from inside to the outside of the metal tubule 10. The metal tubule 10 possesses good heat conduction and is capable of operating under low temperature.

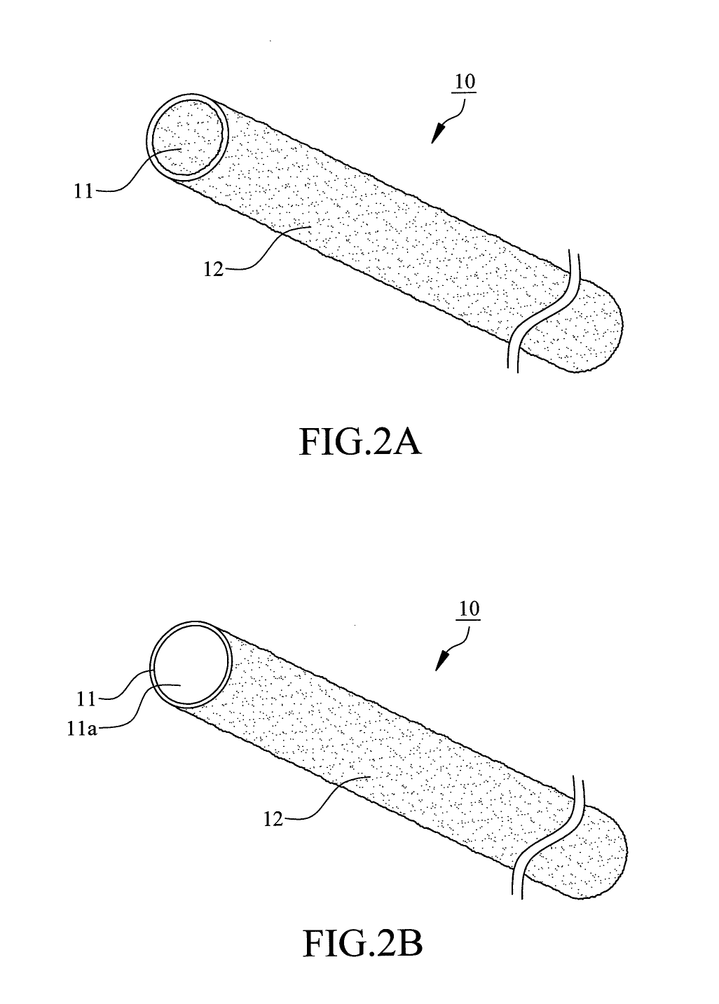

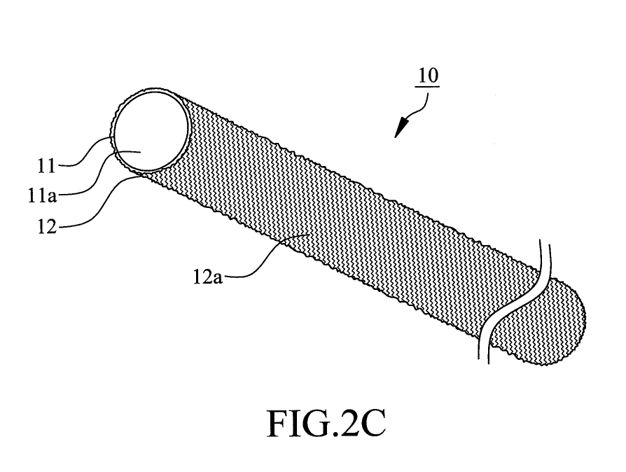

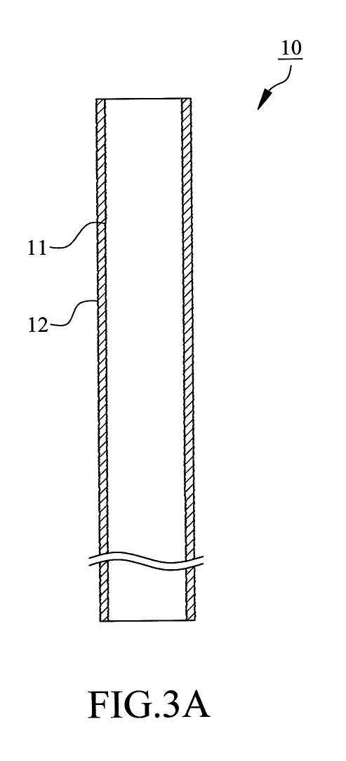

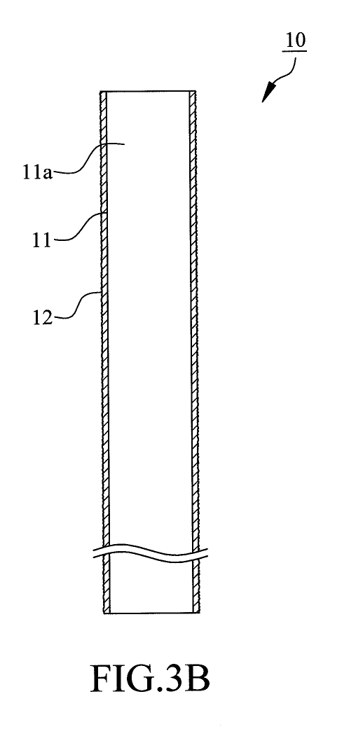

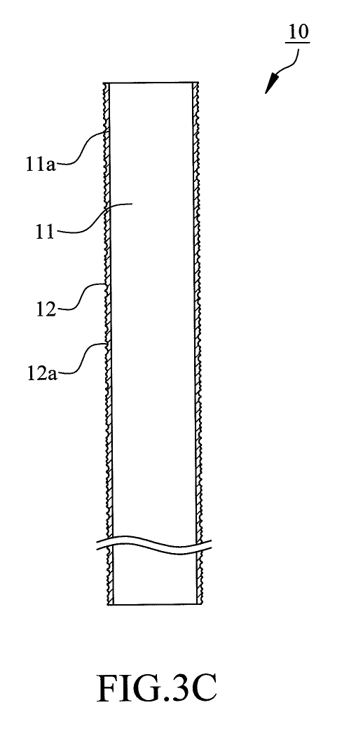

Referring again to FIG. 2C and 3C, wherein a structure of the cell cryopreservation tube is manufactured by the method of this invention. The metal tubule 10 has an inner wall 11 having a smooth surface 11a and an outer wall 12 having a rough surface 12a. In this embodiment, the material of the metal tubule 10 is one of the groups being composed of stainless steel and titanium. The smooth surface 11a has a first roughness, and the rough surface 12a has a second roughness. The magnitude of the second roughness is greater than the first roughness.

While this invention has been particularly illustrated and described in detail with respect to the preferred embodiments thereof, it will be clearly understood by those skilled in the art that is not limited to the specific features shown and described and various modified and changed in form and details may be made without departing from the spirit and scope of this invention.

What is claimed is:

1. A manufacturing method of cell cryopreservation tube comprising the steps of:
(a) Providing a metal tubule having an inner wall and an outer wall;
(b) Smoothing the inner wall of the metal tubule so as to have a smooth surface of the inner wall, and the smooth surface having a first roughness; and
(c) Roughening the outer wall of the metal tubule so as to have a rough surface of the outer wall, and the rough surface having a second roughness, the magnitude of the second roughness is greater than the first roughness.

2. The manufacturing method of cell cryopreservation tube in accordance with claim 1, wherein the method of smoothing the inner wall of the metal tubule is the method of electrolytic polishing.

3. The manufacturing method of cell cryopreservation tube in accordance with claim 1, wherein after the step of smoothing the inner wall of the metal tubule, it further includes a step of sealing two ends of the metal tubule.

4. The manufacturing method of cell cryopreservation tube in accordance with claim 3, wherein the method of roughening the outer wall of the metal tubule is the method of electrochemical anodization treatment.

5. The manufacturing method of cell cryopreservation tube in accordance with claim 1, wherein the method of roughening the outer wall of the metal tubule is the method of grinding.

6. The manufacturing method of cell cryopreservation tube in accordance with claim 1, wherein the material of the metal tubule is one of the groups being composed of stainless steel and titanium.

7. A structure of cell cryopreservation tube is a metal tubule and has an inner wall having a first roughness and an outer wall having a second roughness, the magnitude of the second roughness is greater than the first roughness.

8. The structure of cell cryopreservation tube in accordance with claim 7, wherein the material of the metal tubule is one of the groups being composed of stainless steel and titanium.
